SEQUENCE LISTING (7 sequences)

SEQ ID NO: 1 - protein, 306 aa, Artificial Sequence
  source: /note=“Description of Artificial Sequence Synthetic polypeptide”
MADSDINIKTGTIDIGSNTIVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGATLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTNGLSAWSHPQFEK

SEQ ID NO: 2 - protein, 325 aa, Artificial Sequence
  source: /note=“Description of Artificial Sequence Synthetic polypeptide”
MADSDINIKTGTIDIGSNTIVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTNGGSSGGSSGGAHIVMVDAYKPTKKGHHHHHH

SEQ ID NO: 3 - protein, 867 aa, Artificial Sequence
  source: /note=“Description of Artificial Sequence Synthetic polypeptide”
MHHHHHHHHSGDYDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGGSDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDVEEPKTFHIVQNNNIVYGCNVYMDKFFEVENKDGSTTEIGLCLDFFDSYKIITCAESQFHNYVHDVDPMFYKMGEEYDYDTWRSPTHKQTILELRYQYNDIYMLREVIEQFYIDGLCGGELPLTGMRTASSIAFNVLKKMTFGEEKTEEGYINYFELDKKTKFEFLRKRIEMESYTGGYHANHKAVGKTINKIGCSLDINSAYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTINYNWLTSVEYEFWIKHFNFGVFKKDEYDCFEVDNLEFTGLKIGSILYYKAEKGKFKPYVDHFTKMKVELKKLGNKPLTNQAKLILNGAYGKFGTKQNKEEKDLIMDKNGLLTFTGSVTEYEGKEFYRPYASFVTAYGRLQLWNAIIYAVGVENFLYCDTDSIYCNREVNSLIEDMNAIGETIDKTQLGKWDVEHVFDKFKVLGQKKYMYHDCKEDKTDLKCCGLPSDARKIIIGQGFDEFYLGKNVEGKKQRKKVIGGCLLLDTLFTIKKIMF

SEQ ID NO: 4 - protein, 727 aa, Clostridium phage phiCPV4
MDKHTQYVKEHSFNYDEYKKANFDKIECLIFDTESCTNYENDNTGARVYGWGLGVTRNHNMIYGQNLNQFWEVCQNIFNDWYHDNKHTIKITKTKKGFPKRKYIKFPIAVHNLGWDVEFLKYSLVENGFNYDKGLLKTVFSKGAPYQTVTDVEEPKTFHIVQNNNIVYGCNVYMDKFFEVENKDGSTTEIGLCLDFFDSYKIITCAESQFHNYVHDVDPMFYKMGEEYDYDTWRSPTHKQTTLELRYQYNDIYMLREVIEQFYIDGLCGGELPLTGMRTASSIAFNVLKKMTFGEEKTEEGYINYFELDKKTKFEFLRKRIEMESYTGGYTHANHKAVGKTINKIGCSLDINSSYPSQMAYKVFPYGKPVRKTWGRKPKTEKNEVYLIEVGFDFVEPKHEEYALDIFKIGAVNSKALSPITGAVSGQEYFCTNIKDGKAIPVYKELKDTKLTTNYNVVLTSVEYEFWIKHFNFGVFKKDEYDCFEVDNLEFTGLKIGSILYYKAEKGKFKPYVDHFTKMKVENKKLGNKPLTNQAKLILNGAYGKFGTKQNKEEKDLIMDKNGLLTFTGSVTEYEGKEFYRPYASFVTAYGRLQLWNAIIYAVGVENFLYCDTDSIYCNREVNSLIEDMNAIGETIDKTILGKWDVEHVFDKFKVLGQKKYMYHDCKEDKTDLKCCGLPSDARKIIIGQGFDEFYLGKNVEGKKQRKKVIGGCLLLDTLFTIKKIMF

SEQ ID NO: 5 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 6 - protein, 15 aa, Artificial Sequence
  source: /note=“Description of Artificial Sequence Synthetic peptide”
  MISC_FEATURE (1)..(15): /note=“This sequence may encompass 1-3 ′Gly Gly Gly Gly Ser′ repeating units”
GGGGSGGGGSGGGGS

SEQ ID NO: 7 - protein, 5 aa, Artificial Sequence
  source: /note=“Description of Artificial Sequence Synthetic peptide”
RSKLG

(19) United States
(12) Patent Application Publication
(10) Pub. No.: US 2020/0216887 A1
(43) Pub. Date: Jul. 9, 2020

(54) NANOPORE SEQUENCING COMPLEXES

(71) Applicants: Genia Technologies, Inc., Santa Clara, CA (US); ROCHE DIAGNOSTICS GMBH, Mannheim (DE)

(72) Inventors: Timothy Kellogg Craig, Campbell, CA (US); Christos Tzitzilonis, Redwood City, CA (US); Alexander H. Yang, Campbell, CA (US); Liv E. Jensen, Palo Alto, CA (US); Marshall Porter, Santa Clara, CA (US); Charlotte Yang, San Jose, CA (US); Corissa Harris, Santa Clara, CA (US); Matt Dipetro, Gilroy, CA (US)

(73) Assignee: Genia Technologies, Inc., Santa Clara, CA (US)

(21) Appl. No.: 16/071,406

(22) PCT Filed: Jan. 20, 2017
(86) PCT No.: PCT/EP2017/051219

§ 371 Date: Jul. 19, 2018

Related U.S. Application Data
(60) Provisional application No. 62/281,719, filed on Jan. 21, 2016.

(51) Int. Cl. C12Q 1/6869; C12N 9/12

(52) U.S. Cl. CPC C12Q 1/6869; C12N 9/1247; C12Q 2565/631; C12Q 2521/543; C12N 9/1252

(57) ABSTRACT

A method is provided for preparing nanopore sequencing complexes in membranes for sequencing of polymers, e.g., polynucleotides and polypeptides. The nanopore sequencing complex is formed by the sequential linking of an enzyme to a nanopore that is inserted in a membrane, and of a polymer to the enzyme. Alternatively, the nanopore sequencing complex is formed by linking a preformed enzyme-polymer complex to a nanopore that is inserted in a membrane. The enzyme polymer complex is interchangeable.

### SEQUENCE LISTING

The instant application contains a Sequence Listing which has been submitted in ASCII format via EFS-Web and is hereby incorporated by reference in its entirety. Said ASCII copy, created on May 9, 2019, is named 04338-528US1_SeqlistingCorrected.txt and is 23 kilobytes in size.

### TECHNICAL FIELD

A method is provided for preparing a nanopore sequencing complex in a membrane.

### BACKGROUND

Nanopore analysis is an emerging technique that involves using a voltage to drive molecules through a nanoscale pore in a membrane between two electrolytes, and monitoring how the ionic current through the nanopore changes as single molecules pass through it. This approach allows charged polymers (DNA, RNA, or protein) to be analysed with subnanometer resolution without the need for preprocessing, such as the addition of labels or amplification by polymerase chain reaction.

A nanopore-based diagnostic tool could offer various advantages: it could detect target molecules at very low concentrations from very small sample volumes; it could simultaneously screen panels of biomarkers or genes (which is important in disease diagnosis, monitoring progression and prognosis); it could provide rapid analysis at relatively low cost; and it could eliminate cumbersome amplification and conversion steps such as PCR, bisulphite conversion and Sanger sequencing.

Applications where nanopore technology could excel include whole genome sequencing, microRNA (miRNA) expression profiling, epigenetic analysis, genetic analysis involving single nucleotide polymorphisms, and genomic profiling of viruses and human pathogens.

Improvements in the fabrication of nanopore sensors are needed to scale this technology to an array of 100,000 or more individually addressed nanopores operating in parallel, which would enable sequencing of an entire human genome (some three billion base pairs) with 50-fold coverage in less than one hour (Venkatesan and Bashir, Nature Biotechnol, DOI:10.10381/NNANA.2011.129 [2011]).

### BRIEF SUMMARY OF THE INVENTION

Methods are provided for preparing nanopore sequencing complexes in membranes for sequencing of polymers, e.g., polynucleotides and polypeptides. The nanopore sequencing complex can be formed by sequentially binding to a nanopore that is inserted in a membrane, an enzyme, e.g., a polymerase, followed by the binding of a polymer, e.g., a polynucleotide. Alternatively, the nanopore sequencing complex can be formed by binding a pre-formed enzyme-polymer complex to a nanopore that is inserted in a membrane. The enzyme-polymer complex is interchangeable, i.e., a first enzyme-polymer complex that is bound to a nanopore can be replaced with a second enzyme-polymer complex.

In one embodiment, the method provided for preparing a nanopore sequencing complex comprises (a) inserting a nanopore into a membrane; (b) contacting the nanopore with a sequencing enzyme; (c) attaching the sequencing enzyme to the nanopore to form an enzyme-nanopore complex; and (d) binding a polymer to the enzyme-nanopore complex to provide the nanopore sequencing complex.

In another embodiment, a method is provided for preparing a nanopore sequencing complex that comprises (a) inserting a nanopore into a membrane; (b) preparing an enzyme-polymer complex by associating or binding a sequencing enzyme with a polymer; and (c) attaching the enzyme-polymer complex to the inserted nanopore, thereby providing a nanopore sequencing complex. The enzyme-polymer complex that is attached to the nanopore is an interchangeable complex that can be substituted with any one of a plurality of different enzyme-polymer complexes. In some embodiments, the interchangeable enzyme-polymer complex is an interchangeable polymerase-polynucleotide complex that is substituted with any one of a plurality of different polymerase-polynucleotide complexes. In some embodiments, the polymerase-polynucleotide complex is a DNA polymerase-DNA complex. In other embodiments, the polymerase-polynucleotide complex is a DNA polymerase-DNA complex. In yet other embodiments, the polymerase-polynucleotide complex is an RNA polymerase-RNA complex.

In some embodiments, the sequencing enzyme of the nanopore sequencing complex prepared by any of the methods provided herein, can be a polymerase, an exonuclease, a helicase or an unfoldase. The polymerase can be a DNA polymerase, a reverse transcriptase, or an RNA polymerase. The polymerases can be variants of naturally-occurring polymerases. The variant polymerases can have, for example, increased enzyme activity, fidelity, processivity, elongation rate, stability, or solubility.

In some embodiments, the polymer of the nanopore sequencing complex can be a polynucleotide or a protein. The polynucleotide can be selected from single stranded DNA (ssDNA), double stranded DNA (dsDNA), and RNA. The polynucleotide can be linear, hairpin, or continuous, e.g., circular or dumbbell.

In some embodiments, the nanopore of the nanopore sequencing complex can be selected from a monomeric nanopore, e.g., Outer Membrane Protein G (OMPG), and an oligomeric nanopore, e.g., alpha-hemolysin (a-HL). The oligomeric nanopore can be a homo-oligomeric or a hetero-oligomeric nanopore. The nanopore sequencing complexes can comprise variants and modified variants of the nanopores. The variant or modified variant nanopores can display an altered time to thread (TTT) relative to the parent nanopore. Alternatively, or in addition to the altered time to thread, the variant nanopore can have reduced current noise relative to the parent nanopore.

The nanopore sequencing complex provided according to any of the embodiments of the method enables the sequencing of a polynucleotide or a protein.

In another aspect, a method of sequencing a nucleic acid is provided. The method comprises, (a) preparing a nanopore sequencing complex in a lipid bilayer according to any one of the embodiments of the method for preparing a nanopore-sequencing complex provided herein; (b) providing tagged nucleotides or nucleotide analogs to said nanopore sequencing complex, wherein the tag of said tagged nucleotide is detectable with the aid of said nanopore; (c) carrying out a polymerization reaction with the aid of said polymerase coupled to said nanopore in said nanopore-sequencing complex, thereby incorporating an individual tagged nucleotide of said tagged nucleotides into a growing strand complementary to a sample polynucleotide template associated with or bound to the polymerase; and (d) detecting a tag associated with said individual tagged nucleotide during incorporation of said individual tagged nucleotide, wherein said tag is detected with the aid of said nanopore while said nucleotide is associated with said polymerase, thereby providing a sequence of said nucleic acid sample.

In another aspect, a biochip is provided for sequencing a nucleic acid sample. The biochip can comprise an array of nanopore sequencing complexes disposed in a membrane and prepared according to any one of the embodiments of the methods for preparing a nanopore sequencing complex provided herein. The nanopores of the array are disposed adjacent or in proximity to an electrode, wherein the nanopore is individually addressable and has a single polymerase-polynucleotide complex attached to the nanopore; and wherein an individual nanopore detects the tag associated with the tagged nucleotide during incorporation of the nucleotide into a growing nucleic acid chain by the polymerase.

Other objects, features and advantages of the present invention will become apparent from the following detailed description. It should be understood, however, that the detailed description and specific examples, while indicating preferred embodiments of the invention, are given by way of illustration only, since various changes and modifications within the scope and spirit of the invention will become apparent to one skilled in the art from this detailed description.

### BRIEF DESCRIPTION OF THE DRAWINGS

FIG. 1 depicts three possible nanopore structures that may be inserted into a lipid bilayer from a mixture of (i) nanopore protein, (ii) enzyme, e.g., DNA polymerase, and (iii) substrate template, e.g., DNA: bare nanopore (A), nanopore bound to polymerase (B), and nanopore bound to template-bound polymerase (C).

FIG. 2 depicts a process for preparing a nanopore sequencing complex (iii) on a chip by sequentially adding to a nanopore inserted in a membrane (i) an enzyme that binds to the nanopore to form an enzyme-nanopore complex (ii), and a substrate template, e.g., a DNA template, that associates with the enzyme-nanopore complex to form an enzyme-nanopore-template complex (iii), i.e., a nanopore sequencing complex.

FIG. 3 depicts a process for preparing an enzyme-nanopore-template, i.e., a nanopore sequencing complex (iii), on a chip by flowing a pre-formed enzyme-template complex (ii) on or over a nanopore that is inserted into the lipid bilayer of a chip (i). Reference is made to Example 1.

FIG. 4 depicts single channel current traces at an applied voltage across a nanopore sequencing complex in a lipid membrane. Reference is made to Example 1.

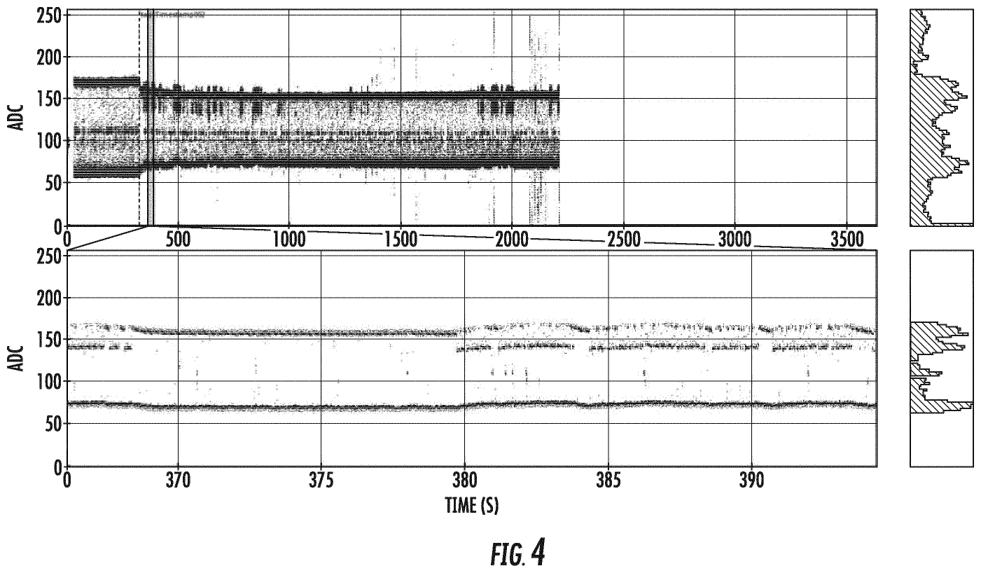

The file of this patent contains at least one drawing in color. Copies of this patent or patent publication with color drawing(s) will be provided by the Office upon request and payment of the necessary fee.

### DETAILED DESCRIPTION

A detailed description of one or more embodiments of the invention is provided below along with accompanying figures that illustrate the principles of the invention. The invention is described in connection with such embodiments, but the invention is not limited to any single embodiment. The scope of the invention is limited only by the claims, and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

All patents and publications, including all sequences disclosed within such patents and publications, referred to herein are expressly incorporated by reference.

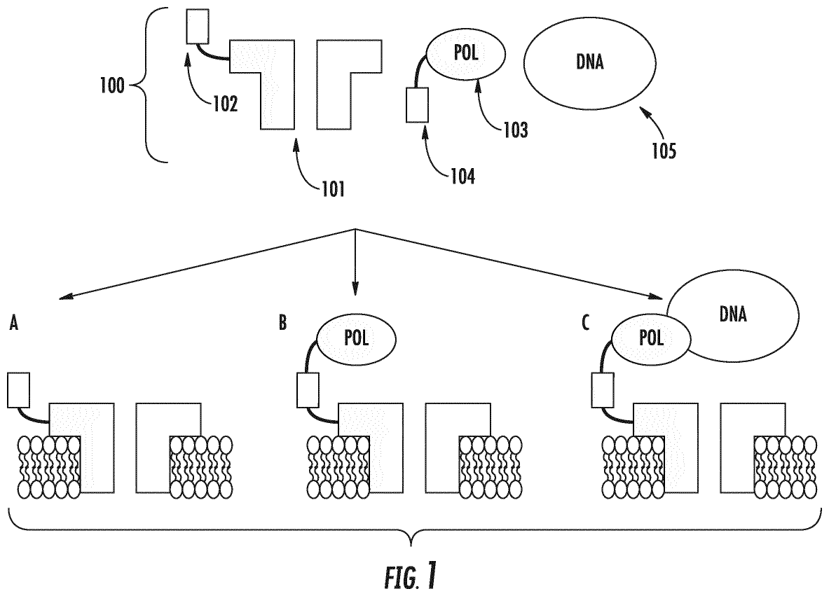

Current methods for providing an enzyme-nanopore-polymer complex comprise incubating nanopore protein, polymer, and enzyme to allow association of the components, flowing the reaction mixture onto a membrane, and either actively or passively reconstituting the resulting structures into the membrane. The three possible structures that become inserted into the membrane are: the nanopore alone, the nanopore associated with the enzyme, and the nanopore associated with the enzyme and polymer. For example, as shown in FIG. 1, flowing a reaction mixture (100) of nanopore (101) comprising an attachment component (102), polymerase enzyme (Pol) (103) comprising an attachment component (104), and polynucleotide (105) onto a lipid bilayer under conditions that will cause the nanopore to insert into the bilayer, e.g., electroporation, will result in (A) the nanopore alone being inserted into the bilayer, (B) the nanopore associated with the polymerase (enzyme-nanopore complex) being inserted into the bilayer, and (C) the nanopore-polymerase-polynucleotide template, e.g., DNA, complex being inserted into the bilayer. Electroporation efficiency, i.e., the efficiency whereby diffusion of nanopore-polymerase-polynucleotide complexes are inserted into a membrane, depends on the size of the nanopore complex. The larger the complex, the less the electroporation efficiency. Thus, of the three possible structures shown in FIG. 1, the nanopore alone (A) will insert more readily into the membrane than (B) nanopore-polymerase complex, which in turn, will insert more readily than (C) the nanopore-polymerase-polynucleotide complex. Structures A and B are non-functional as neither comprises all three components that are necessary to determine the sequence of the polymer, e.g., DNA. Additionally, A and B will occupy space on a biochip thereby diminishing the density of functional enzyme-nanopore-polymer complexes on a biochip.

Alternatively, current methods can comprise pre-forming the nanopore sequencing complex, and inserting it into the membrane. However, pre-forming the nanopore-sequencing complex is inefficient and costly as it requires that the nanopore sequencing complex be purified to a high degree prior to being inserted into the membrane.

Provided herein is a method for preparing a nanopore-sequencing complex that overcomes the limitations of current processes. The method comprises inserting a nanopore into a membrane, preparing an enzyme-polymer complex, and attaching the enzyme-polymer complex to the inserted pore. The current method ensures that only the complete nanopore sequencing complex, i.e., complex having the requisite components required for determining polymer sequences, is inserted into the membrane, resulting in high occupancy by nanopore sequencing complexes, thereby maximizing sequencing output.

Additionally, the method set forth herein is advantageous for creating nanopore sequencing complexes having interchangeable enzyme-polymer complexes. Any one type of enzyme-polymer complex that is coupled to a nanopore inserted into a membrane can be exchanged for any one of a plurality of different enzyme-polymer complexes. For example, a sample polynucleotide template that associated with a first polymerase, e.g., T7 DNA polymerase, as a first enzyme polymer complex bound to an aHL nanopore, can be substituted by a sample polynucleotide template that is associated with a second polymerase, e.g., Pol II polymerase, as a second enzyme polymer complex, which replaces the first enzyme-polymer complex. Any one of the polymer in the polymer enzyme complex, the enzyme in the polymer enzyme complex, and the enzyme polymer complex itself can be replaced in the nanopore sequencing complex.

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham. THE HARPER COLLINS DICTIONARY OF BIOLOGY, Harper Perennial, N.Y. (1991) provide one of skill with a general dictionary of many of the terms used in this invention. Although any methods and materials similar or equivalent to those described herein can be used in the practice or testing of the present invention, the preferred methods and materials are described. Practitioners are particularly directed to Sambrook et al., 1989, and Ausubel F M et al., 1993, for definitions and terms of the art. It is to be understood that this invention is not limited to the particular methodology, protocols, and reagents described, as these may vary.

Numeric ranges are inclusive of the numbers defining the range. The term about is used herein to mean plus or minus ten percent (10%) of a value. For example, “about 100” refers to any number between 90 and 110.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention, which can be had by reference to the specification as a whole. Accordingly, the terms defined immediately below are more fully defined by reference to the specification as a whole.

### Definitions

The terms “polynucleotide” and “nucleic acid” are herein used interchangeably to refer to a polymer molecule composed of nucleotide monomers covalently bonded in a chain. Single stranded DNA (ss deoxyribonucleic acid; ssDNA), double stranded DNA (dsDNA) and RNA (ribonucleic acid) are examples of polynucleotides.

The term “amino acid” in its broadest sense, herein refers to any compound and/or substance that can be incorporated into a polypeptide chain. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, and/or substitution with other chemical without adversely affecting their activity, Amino acids may participate in a disulfide bond. The term “amino acid” is used interchangeably with “amino acid residue,” and may refer to a free amino acid and/or to an amino acid residue of a peptide. It will be apparent from the context in which the term is used whether it refers to a free amino acid or a residue of a peptide. It should be noted that all amino acid residue sequences are represented herein by formulae whose left and right orientation is in the conventional direction of amino-terminus to carboxy-terminus.

The term “nanopore sequencing complex” herein refers to a nanopore linked to an enzyme, e.g., a polymerase, exonuclease, unfoldase, etc., which in turn is associated with a polymer, e.g., a polynucleotide or a protein. The nanopore sequencing complex is positioned in a membrane, e.g., a lipid bilayer, where it functions to identify polymer components, e.g., nucleotides or amino acids.

The term “enzyme-polymer complex” herein refers to an enzyme, e.g., polymerase, exonuclease, unfoldase, etc., that is associated/coupled with a polymer, e.g., polynucleotide or protein.

The term “enzyme-nanopore complex” herein refers to a nanopore that is associated/coupled with a sequencing enzyme. In some embodiments, the nanopore can be reversibly or irreversibly bound to the sequencing enzyme.

The term “nucleotide” herein refers to a monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. When the nucleoside contains a phosphate group bonded to the 3′ or 5′ position of the pentose it is referred to as a nucleotide. A sequence of operatively linked nucleotides is typically referred to herein as a “base sequence” or “nucleotide sequence,” and is represented herein by a formula whose left to right orientation is in the conventional direction of 5′-terminus to 3′-terminus.

The term “nucleotide analog” herein refers to analogs of nucleoside triphosphates, e.g., (S)-Glycerol nucleoside triphosphates (gNTPs) of the common nucleobases: adenine, cytosine, guanine, uracil, and thymidine (Horhota et al. Organic Letters, 8:5345-5347 [2006]).

The term “tag” herein refers to a detectable moiety that may be atoms or molecules, or a collection of atoms or molecules. A tag may provide an optical, electrochemical, magnetic, or electrostatic (e.g., inductive, capacitive) signature, which may be detected with the aid of a nanopore.

The term “tagged nucleotide” herein refers to a nucleotide having a tag attached at its terminal phosphate.

The term “sequencing enzyme” herein refers to the enzyme of a nanopore sequencing complex where it serves to identify polymer components, e.g., nucleotides or amino acids.

The term “polymerase” herein refers to an enzyme that catalyzes the polymerization of nucleotide (i.e., the polymerase activity). The term polymerase encompasses DNA polymerases, RNA polymerases, and reverse transcriptases. -A “DNA polymerase” catalyzes the polymerization of deoxynucleotides. An “RNA polymerase” catalyzes the polymerization of ribonucleotides. A “reverse transcriptase” catalyzes the polymerization of deoxynucleotides that are complementary to an RNA template.

The terms “template DNA molecule” and “template strand” are used interchangeably herein to refer to a strand of a nucleic acid from which a complementary nucleic acid strand is synthesized by a DNA polymerase, for example, in a primer extension reaction.

The term “sample polynucleotide” herein refers to a polynucleotide obtained from a sample, e.g., a biological sample.

The term “template-dependent manner” refers to a process that involves the template dependent extension of a primer molecule (e.g., DNA synthesis by DNA polymerase). The term “template-dependent manner” typically refers to polynucleotide synthesis of RNA or DNA wherein the sequence of the newly synthesized strand of polynucleotide is dictated by the well-known rules of complementary base pairing (see, for example, Watson, J. D. et al., In: Molecular Biology of the Gene, 4th Ed., W. A. Benjamin, Inc., Menlo Park, Calif. (1987)).

The term “nanopore” herein refers to a channel or passage formed or otherwise provided in a membrane. A membrane may be an organic membrane, such as a lipid bilayer, or a synthetic membrane, such as a membrane formed of a polymeric material. The nanopore may be disposed adjacent or in proximity to a sensing circuit or an electrode coupled to a sensing circuit, such as, for example, a complementary metal oxide semiconductor (CMOS) or field effect transistor (FET) circuit. In some examples, a nanopore has a characteristic width or diameter on the order of 0.1 Nm to about 1000 nm. Some nanopores are proteins. OmpG and alpha-hemolysin are examples of a protein nanopore.

The term “monomeric nanopore” herein refers to a nanopore protein that consists of a single subunit. OmpG is an example of a monomeric nanopore.

The term “oligomeric nanopore” herein refers to nanopores that can be composed of multiple identical subunits, multiple distinct subunits, or a mixture of identical and distinct subunits. Nanopores with identical subunits are termed “homo-oligomeric nanopores”. Nanopores containing two or more distinct polypeptide subunits are termed “hetero-oligomeric nanopores”. Alpha-hemolysin is an example of an oligomeric nanopore.

The term “wild-type” herein refers to a gene or gene product (e.g., a protein) that has the characteristics of that gene or gene product when isolated from a naturally occurring source.

The term “parental” or “parent” herein refers to a protein, e.g., a nanopore or enzyme, to which modifications, e.g., substitution(s), insertion(s), deletion(s), and/or truncation(s), are made to produce variants thereof. This term also refers to the polypeptide with which a variant is compared and aligned. The parent may be a naturally occurring (wild type) polypeptide, or it may be a variant thereof, prepared by any suitable means.

The term “mutation” herein refers to a change introduced into a parental sequence, including, but not limited to, substitutions, insertions, deletions (including truncations). The consequences of a mutation include, but are not limited to, the creation of a new character, property, function, phenotype or trait not found in the parental sequence.

The term “variant” herein refers to a modified protein which displays altered characteristics when compared to the parental protein, e.g., altered ionic conductance, altered time to thread, etc.

The term “modified variant” herein refers to a variant protein that has been further modified to alter its physical interaction with other proteins. For example, a modified variant protein may be a multimeric variant nanopore that has been further mutated to affect inter-subunit interactions. For example, a modified variant protein may be a multimeric variant nanopore e.g., a variant aHL nanopore that has been mutated to alter a characteristic of the oligomerized nanopore e.g., “time to thread”, and that additionally has a mutation in one or more of its subunits that alters the interaction of the subunits and hence the oligomerization of the nanopore.

The term “time to thread” or “TTT” herein refers to the time it takes the polymerase-tag complex to thread the tag into the nanopore.

The terms “noise” and “ionic current noise” are herein used interchangeably and refer to random fluctuations of electrical signal, which include current fluctuations contributed by spontaneous gating and current fluctuations contributed by the inherent architecture of the nanopore. For example, the tertiary make-up of the nanopore can comprise more than one recognition site for the analyte that is being sensed by the nanopore thereby inducing additional signals that contribute to the overall noise of the channel.

The term “constriction amino acids” herein refers to the amino acids that determine the size of a nanopore at the constriction zone. The constriction zone may be the same as the constriction zone of the wild-type nanopore or it may be a constriction zone introduced via protein engineering, or by the introduction of a molecular adapter.

The terms “alpha-hemolysin,” “α-hemolysin,” “aHL,” “αHL,” “a-HL” and “α-HL” are used interchangeably and herein refer to a protein that self-assembles into a heptameric water-filled transmembrane channel from monomers, concatemers of monomers, or a combination of monomers and concatemers of monomers.

The term “purified” herein refers to a polypeptide that is present in a sample at a concentration of at least 95% by weight, or at least 98% by weight of the sample in which it is contained.

The term “attachment component” herein refers to a structure, e.g., a linker, which attaches an enzyme-polymer complex to a nanopore in a membrane.

### Nanopore Sequencing Complexes

In one aspect, the disclosure provides a method for preparing a nanopore sequencing complex in situ, i.e., on a biochip.

In some embodiments, the method comprises adding the components of the nanopore sequencing complex in a step-wise manner. Accordingly, in some embodiments, the method comprises inserting a nanopore into a membrane, e.g., a lipid bilayer, contacting the nanopore with a sequencing enzyme, e.g., DNA polymerase, to attach the sequencing enzyme to the nanopore to form an enzyme-nanopore complex, and subsequently contacting the enzyme-nanopore complex with a polymer, e.g., DNA, to form a nanopore sequencing complex.

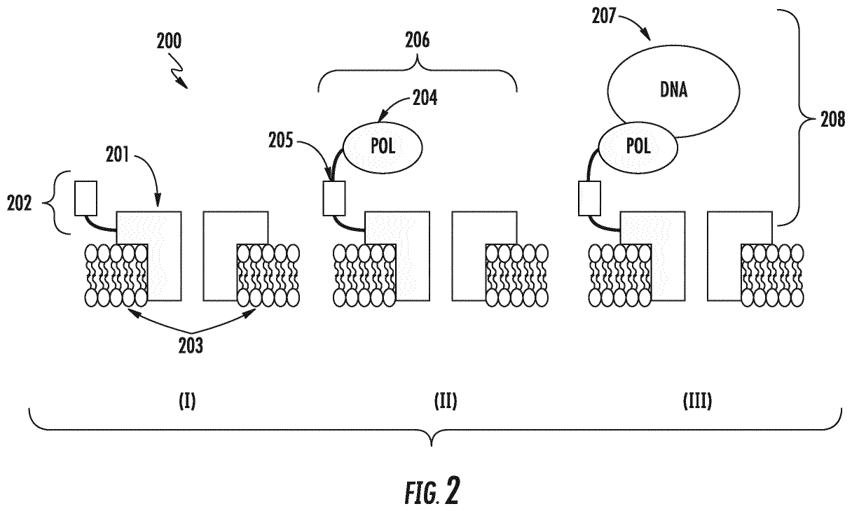

An exemplary method for preparing a nanopore sequencing complex by step-wise addition of its components is shown in FIG. 2.

FIG. 2 depicts a method (200) for preparing a nanopore sequencing complex that comprises a nanopore, a DNA polymerase and DNA. The method comprises: (i) inserting a nanopore (201) having an attachment component (202) into a membrane, e.g., a lipid bilayer (203); (ii) contacting the nanopore with a sequencing enzyme. e.g., a polymerase enzyme (204) having an attachment component (205), to attach the sequencing enzyme to the nanopore and thereby form an enzyme-nanopore complex (206), and (iii) contacting the enzyme-nanopore complex with a polymer, e.g., DNA (207) to form a nanopore sequencing complex (208).

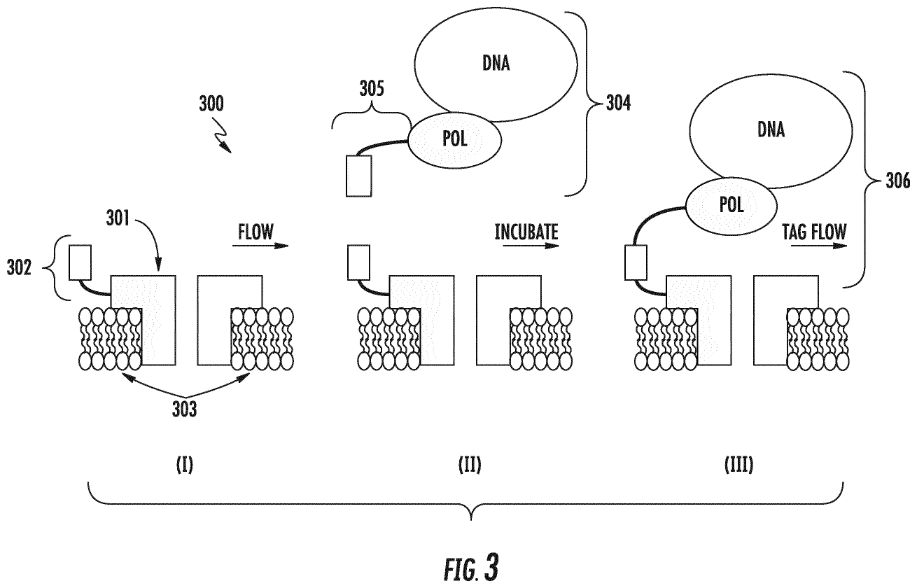

In other embodiments, the method comprises pre-forming an enzyme-polymer complex. Pre-forming the enzyme-polymer complex comprises combining a sequencing enzyme with a polymer to enable the binding of the polymer with the enzyme. The pre-formed enzyme-polymer complex is subsequently attached to the nanopore that is inserted in a membrane to form the nanopore sequencing complex. The nanopore sequencing complex enables sequencing of polymers, e.g., polynucleotides and polypeptides. Accordingly, in some embodiments, the sequencing enzymes may be enzymes that are coupled to polynucleotides, and that enable nanopore sensing of polynucleotide components, i.e., nucleotide bases, tags of nucleotide bases, and/or nucleotide analogs. In other embodiments, the sequencing enzymes may be enzymes that are coupled to polypeptides, and that enable nanopore sensing of polypeptide components, i.e., amino acids or analogs thereof. An example of a method for preparing a nanopore sequencing complex that comprises attaching a preformed complex to the nanopore is shown in FIG. 3.

FIG. 3 depicts a method (300) for preparing a nanopore sequencing complex that comprises attaching a pre-formed DNA polymerase-DNA complex to a nanopore inserted in a lipid membrane. The method comprises: (i) inserting a nanopore (301) having an attachment component (302) into a membrane, e.g., a lipid bilayer (303); (ii) incubating a pre-formed enzyme-polymer complex, e.g., a polymerase-DNA (304), having an attachment component (305), with the inserted nanopore to attach the enzyme-polymer complex to the nanopore via interaction between the attachment components of the enzyme-polymer complex and the nanopore (305 and 301, respectively), thereby forming (iii) a nanopore sequencing complex (306). Sequencing of the DNA template can be performed by detecting nucleotides, e.g., tagged nucleotides, or tags of tagged nucleotides as the nucleotide babes are incorporated by the polymerase into a DNA strand that is complementary to that of the DNA template.

Sequencing enzymes of the nanopore sequencing complexes can be polymerases, exonucleases, helicases, or unfoldases. The polymerases can be DNA polymerases, reverse transcriptases or RNA polymerases. Sequencing enzymes are described elsewhere herein.

Polymers of the nanopore sequencing complexes can be polynucleotides or proteins. The polynucleotides can be single stranded DNA (ssDNA), double stranded DNA (dsDNA), or RNA. The polynucleotides can be obtained from a variety of biological samples including but not limited to tissue samples, biological fluid samples, or a cell samples, and processed fractions thereof.

Nanopores of the nanopore sequencing complexes can be monomeric nanopores, e.g., Outer Membrane Protein G (OMPG), or oligomeric nanopores, e.g., alpha-hemolysin (a-HL). Oligomeric nanopores can be homo-oligomeric nanopore, and a hetero-oligomeric nanopore, as described elsewhere herein.

### Enzyme-Polymer Complexes

In some embodiments, the polymer component of the enzyme polymer complex is a sample template polynucleotide, e.g. DNA or RNA, and the enzyme of the enzyme polymer complex is a polymerase, a DNA packaging enzyme, a terminase, or an exonuclease. DNA may be single stranded or double stranded, and it may be genomic, i.e., nuclear DNA, mitochondrial DNA, or cell-free DNA (cfDNA). RNA may be ribosomal RNA (rRNA), messenger RNA (mRNA), micro RNA (miRNA), or small interfering RNA (siRNA). The nucleotide may be circular.

In some embodiments, the sequencing enzyme-polymer complex and of the enzyme-nanopore complex enables nanopore sequencing of DNA. In some embodiments, the polynucleotide of the enzyme polymer complex is DNA, and the enzyme of the enzyme -polymer complex and of the enzyme-nanopore complex is a polymerase, e.g., a DNA polymerase. In other embodiments, the polynucleotide of the enzyme polymer complex is DNA and the enzyme of the enzyme-DNA complex is an RNA polymerase. In other embodiments, the polynucleotide of the enzyme polymer complex is DNA, and the enzyme of the enzyme-DNA complex is an exonuclease. In yet other embodiments, the polynucleotide of the enzyme polymer complex is DNA, and the enzyme of the enzyme-DNA complex is a helicase.

In another aspect, the enzyme-polymer complex enables nanopore sequencing of RNA. In some embodiments, the polynucleotide of the enzyme polymer complex is RNA, and the enzyme of the enzyme-polymer complex is a polymerase, e.g., a reverse transcriptase. In other embodiments, the enzyme of the enzyme-RNA complex is an exonuclease. In yet other embodiments, the enzyme of the enzyme-RNA complex is an RNA-binding ATPase.

Nanopore sequencing of DNA and RNA may be achieved by strand sequencing and/or exosequencing of DNA and RNA. Strand sequencing comprises methods whereby nucleotide bases of a sample polynucleotide strand are determined directly as the nucleotides of the polynucleotide template are threaded through the nanopore. Alternatively, strand sequencing of the polynucleotide strand determines the sequence of the template indirectly by determining nucleotides that are incorporated into a growing strand that is complementary to that of the sample template strand.

In some embodiments, DNA, e.g., single stranded DNA, may be sequenced by detecting tags of tagged nucleotides that are released from the nucleotide base as the nucleotide is incorporated by a polymerase into a strand complementary to that of a template associated with the polymerase in an enzyme-polymer complex. The single molecule nanopore-based sequencing by synthesis (Nano-SBS) technique that uses tagged nucleotides is described, for example, in PCT/US2013/068967 (published as WO2014/074727, Genia Technologies, Inc.).

Accordingly, in some embodiments, the enzyme-polynucleotide complex that may be attached to the inserted nanopore may be a DNA polymerase-DNA complex. In some embodiments, the DNA polymerase-DNA complex may be attached to a wild-type or variant monomeric nanopore. In some embodiments, the DNA polymerase-DNA complex may be attached to a wild-type, variant, or modified variant homo-oligomeric nanopore. In some embodiments, the DNA polymerase-DNA complex may be attached to a wild-type, a variant, or a modified variant hetero-oligomeric nanopore. In some embodiments, the DNA polymerase-DNA complex may be attached to a wild-type, variant, or modified variant aHL nanopore. In other embodiments, the DNA polymerase-DNA complex may be attached to a wild-type OmpG nanopore or variants thereof.

In other embodiments, the enzyme-polynucleotide complex may be an RNA polymerase-RNA complex. The RNA polymerase-RNA complex may be attached to a wild-type or variant oligomeric or monomeric nanopore. In some embodiments, the RNA polymerase-RNA complex is attached to a wild-type or variant OmpG nanopore. In other embodiments, the RNA polymerase-RNA complex is attached to a wild-type or variant aHL nanopore. In yet other embodiments, the enzyme-polynucleotide complex may be a reverse transcriptase-RNA complex. The reverse transcriptase-RNA complex may be attached to a wild-type or variant oligomeric or monomeric nanopore. In some embodiments, the reverse transcriptase-RNA complex is attached to a wild-type or variant OmpG nanopore. In other embodiments, the reverse transcriptase-RNA complex is attached to a wild-type or variant aHL nanopore.

In some embodiments, individual nucleic acids may be sequenced by the identification of nucleoside 5′-monophosphates as they are released by processive exonucleases (Astier et al., J Am Chem Soc 128:1705-1710 [2006]). Accordingly, in some embodiments, the enzyme-polynucleotide complex that may be attached to the inserted nanopore may be an exonuclease-polynucleotide complex. In some embodiments, the exonuclease-polynucleotide complex may be attached to a wild-type or variant monomeric nanopore. In some embodiments, the exonuclease-polynucleotide complex may be attached to a wild-type or variant homo-oligomeric nanopore. In some embodiments, the exonuclease-polynucleotide complex may be attached to a wild-type or variant hetero-oligomeric nanopore. In some embodiments, the exonuclease-polynucleotide complex may be attached to a wild-type aHL nanopore or variants thereof. In other embodiments, the exonuclease-polynucleotide complex may be attached to a wild-type OmpG nanopore or variants thereof.

Proteins and peptides have also been shown to move through nanopores, and sequencing of a protein using a nanopore can be performed by controlling the unfolding and translocation of the protein through the nanopore. The controlled unfolding and subsequent translocation can be achieved by the action of an unfoldase enzyme coupled to the protein to be sequenced (Nivala et al., Nature Biotechnol 31:247-250 [2013]).

Thus, in some embodiments, the enzyme-polymer complex that is attached to the nanopore in the membrane may be an enzyme-polypeptide complex, e.g., an unfoldase-protein complex. In some embodiments, the unfoldase-protein complex may be attached to a wild-type or variant monomeric nanopore. In some embodiments, the unfoldase-protein complex may be attached to a wild-type or variant homo-oligomeric nanopore. In some embodiments, the unfoldase-protein complex may be attached to a wild-type or variant hetero-oligomeric nanopore. In some embodiments, the unfoldase-protein complex may be attached to a wild-type aHL nanopore or variants thereof. In other embodiments, the unfoldase-protein complex may be attached to a wild-type OmpG nanopore or variants thereof.

### Enzymes of Enzyme-Polymer Complexes and Enzyme-Nanopore Complexes

The enzymes of the enzyme-polymer complex and enzyme-nanopore complex include polynucleotide and polypeptide processing enzymes, e.g. DNA and RNA polymerases, reverse transcriptases, exonucleases, and unfoldases. The enzyme of the enzyme-polymer complex and enzyme-nanopore complex can be a wild-type enzyme, or it can be a variant form of the wild-type enzyme. Variant enzymes can be engineered to possess characteristics that are altered relative to those of the parent enzyme. In some embodiments, the enzyme of the enzyme-polymer complex that is altered is a polymerase. The altered characteristics of the polymerase enzyme include changes in enzyme activity, fidelity, processivity, elongation rate, stability, or solubility. The polymerase can be mutated to reduce the rate at which the polymerase incorporates a nucleotide into a nucleic acid strand (e.g., a growing nucleic acid strand). The reduced velocities (and improved sensitivities) can be achieved by a combination of site-specific mutagenesis of the nanopore protein and the incorporation of DNA processing enzymes, e.g., DNA polymerase, into the nanopore.

In some cases, the rate at which a nucleotide is incorporated into a nucleic acid strand can be reduced by functionalizing the nucleotide and/or template strand to provide steric hindrance, such as, for example, through methylation of the template nucleic acid strand. In some instances, the rate is reduced by incorporating methylated nucleotides.

The enzymes of the enzyme-polymer complex and enzyme-nanopore complex may be modified to comprise one or more attachment components and/or attachment sites that serve to link the enzyme-polymer complex to the nanopore inserted into the membrane. Similarly, the nanopore of the enzyme-nanopore complex and the nanopore to which the enzyme-polymer complex is attached may also be modified to comprise one or more attachment components and/or attachment sites to link the nanopore to the enzyme-polymer complex.

### Nanopores

The nanopores of the nanopore sequencing complex include without limitation biological nanopores, solid state nanopores, and hybrid biological-solid state nanopores. Biological nanopores of the nanopore sequencing complexes include OmpG from E. coli, sp., Salmonella sp., Shigella sp., and Pseudomonas sp., and alpha hemolysin from S. aureus sp., MspA from M. smegmatis sp. The nanopores may be wild-type nanopores, variant nanopores, or modified variant nanopores.

Variant nanopores can be engineered to possess characteristics that are altered relative to those of the parental enzyme. See, for example, U.S. patent application Ser. No. 14/924,861 filed Oct. 28, 2015, entitled “alpha-Hemolysin Variants with Altered Characteristics”. In some embodiments, the characteristics are altered relative to the wild-type enzyme. In some embodiments, the variant nanopore of the nanopore sequencing complex is engineered to reduce the ionic current noise of the parental nanopore from which it is derived. An example of a variant nanopore having an altered characteristic is the OmpG nanopore having one or more mutations at the constriction site (U.S. Provisional Patent Application No. 62/222,197, entitled “OmpG Variants”, filed on Sep. 22, 2015, which is incorporated by reference herein in its entirety), which decrease the ionic noise level relative to that of the parent OmpG. The reduced ionic current noise provides for the use of these OmpG nanopore variants in single molecule sensing of polynucleotides and proteins. In other embodiments, the variant OmpG polypeptide can be further mutated to bind molecular adapters, which while resident in the pore slow the movement of analytes, e.g., nucleotide bases, through the pore and consequently improve the accuracy of the identification of the analyte (Astier et al., J Am Chem Soc 10.1021/ja057123+, published online on Dec. 30, 2005).

Modified variant nanopores are typically multimeric nanopores whose subunits have been engineered to affect inter-subunit interaction (U.S. Provisional Patent Application Nos. 62/232,175 and 62/244,852, entitled “Alpha-Hemolysin Variants”, filed on Sep. 24, 2015 and Oct. 22, 2015, respectively, which are incorporated by reference herein in its entirety). Altered subunit interactions can be exploited to specify the sequence and order with which monomers oligomerize to form the multimeric nanopore in a lipid bilayer. This technique provides control of the stoichiometry of the subunits that form the nanopore. An example of a multimeric nanopore whose subunits can be modified to determine the sequence of interaction of subunits during oligomerization is an aHL nanopore.

### Means of Attaching

The enzyme-polymer complex, e.g., polymerase-polynucleotide complex, can be attached to the nanopore in any suitable way. Attaching enzyme-polymer complexes to nanopores may be achieved using the SpyTag/SpyCatcher peptide system (Zakeri et al. PNAS 109:E690-E697 [2012]) native chemical ligation (Thapa et al., Molecules 19:14461-14483 [2014]), sortase system (Wu and Guo, J Carbohydr Chem 31:48-66 [2012]; Heck et al., Appl Microbiol Biotechnol 97:461-475 [2013]), transglutaminase systems (Dennler et al, Bioconjug Chem 25:569-578 [2014]), formylglycine linkage (Rashidian et al., Bioconjug Chem 24:1277-1294 [2013]), or other chemical ligation techniques known in the art.

In some instances, the polymerase is linked to the nanopore using Solulink™ chemistry. Solulink™ can be a reaction between HyNic (6-hydrazino-nicotinic acid, an aromatic hydrazine) and 4FB (4-formylbenzoate, an aromatic aldehyde). In some instances, the polymerase is linked to the nanopore using Click chemistry (available from LifeTechnologies, for example).

In some cases, zinc finger mutations are introduced into the hemolysin molecule and then a molecule is used (e.g., a DNA intermediate molecule) to link the polymerase to the zinc finger sites on the hemolysin.

Additionally, enzyme-polymer complexes, e.g., polymerase-DNA complexes, can be attached to a nanopore, e.g., aHL, OmpG, by means of a linker molecule that is attached to a nanopore at an attachment site. In some cases, the polymerase-DNA complex is attached to the nanopore with molecular staples. In some instances, molecular staples comprise three amino acid sequences (denoted linkers A, B and C). Linker A can extend from a nanopore monomer, Linker B can extend from the polymerase of the polymerase-DNA complex, and Linker C then can bind Linkers A and B (e.g., by wrapping around both Linkers A and B) and thus the polymerase of the polymerase-DNA complex to the nanopore. Linker C can also be constructed to be part of Linker A or Linker B, thus reducing the number of linker molecules.

Other linkers that may find use in attaching the polymerase to a nanopore are direct genetic linkage (e.g., (GGGGS)₁₋₃ amino acid linker (SEQ ID NO: 6)), transglutaminase mediated linking (e.g., RSKLG (SEQ ID NO: 7)), sortase mediated linking, and chemical linking through cysteine modifications. Specific linkers contemplated as useful herein are (GGGGS)₁₋₃ (SEQ ID NO: 6), K-tag (RSKLG (SEQ ID NO: 7)) on N-terminus, ΔTEV site (12-25), ΔTEV site+N-terminus of SpyCatcher (12-49).

An exemplary method for attaching a polymerase-polynucleotide complex to a nanopore in a membrane involves attaching a linker molecule to a nanopore or mutating a nanopore to have an attachment site and then attaching a polymerase-polynucleotide complex to the attachment site or attachment linker. The polymerase-polynucleotide complex is attached to the attachment site or attachment linker after the nanopore is inserted in the membrane. In some cases, a polymerase-polynucleotide complex is attached to each of a plurality of nanopores that are inserted into a membrane and disposed over wells and/or electrodes of a biochip.

In some instances, the enzyme of the enzyme-polymer complex is expressed as a fusion protein that comprises a linker peptide. In some embodiments, a polymerase is the enzyme of the enzyme-polymer complex, and a polynucleotide is the polymer. The polymerase of the polymerase-polynucleotide complex is expressed as a fusion protein that comprises a SpyCatcher polypeptide, which can be covalently bound to a nanopore that comprises a SpyTag peptide (Zakeri et al. PNAS 109:E690-E697 [2012]).

Polymerase-polynucleotide complexes may be attached using methods described, for example, in PCT/US2013/068967 (published as WO2014/074727; Genia Technologies, Inc.), PCT/US2005/009702 (published as WO2006/028508; President and Fellows of Harvard College), and PCT/US2011/065640 (published as WO2012/083249; Columbia University).

As the rate of formation of the nanopore sequencing complex is dependent on the concentration of the reactants, e.g., the concentration of nanopore and the concentration of the enzyme-polymer complex, it was surprising that the nanopore sequencing complex could be formed in situ as described herein, as the nanopore concentration is extremely small, e.g., one nanopore per well.

### Stoichiometries

The enzymes of the enzyme-polymer complex, e.g., a DNA polymerase, may be modified to comprise one or more attachment components and/or attachment sites that serve to link the enzyme-polymer complex to the nanopore inserted into the membrane. Similarly, the nanopore to which the enzyme polymer complex is attached may also be modified to comprise one or more attachment components and/or attachment sites to link the nanopore to the enzyme-polymer complex.

In some embodiments, a single enzyme-polymer complex is attached to the nanopore in the membrane. In other embodiments, two or more enzyme-polymer complexes are attached to the nanopore in the membrane. The single enzyme-polymer complex may be attached to the nanopore at one or more attachment sites present on the nanopore protein. In some embodiments, one or more enzyme-polymer complexes are attached to a monomeric nanopore protein, e.g., an OmpG nanopore. In other embodiments, one or more enzyme-polymer complexes are attached to a multimeric nanopore protein that comprises at least two nanopore subunits, e.g., heptameric aHL nanopore. An enzyme-polymer complex can be attached to an attachment site on a single subunit of a multimeric nanopore. Alternatively, the enzyme-polymer complex may be attached to the multimeric nanopore at two or more attachments sites present on each of two or more subunits of a multimeric nanopore.

Enzyme-polymer complexes may be attached to one or more subunits of homo-oligomeric or of hetero-oligomeric nanopores. For example, an enzyme-polymer complex may be coupled to an aHL nanopore consisting of 7 identical subunits, which may be wild-type, variants, or modified variants. Alternatively, the enzyme-polymer complex may be coupled to an aHL nanopore consisting of at least two different subunits, of which one or more may be modified.

Oligomeric nanopores, e.g., a-HL, are proteins that can self-assemble from subunits that are monomers, concatemers of monomers, or a combination of monomers and concatemers of monomers. Subunits that are concatemers of monomers can comprise two, three or more monomers that are linked to each other by a linker or that are each encoded by a single polynucleotide as a fusion protein. Accordingly, the enzyme-polymer complex may be attached to a monomer subunit, or to a concatemer of monomer subunits of an oligomeric nanopore. In some embodiments, the enzyme-polymer complex is a DNA polymerase-DNA template complex that is attached to a monomeric nanopore, e.g., an OmpG, which may be wild type, or a variant having altered characteristics. In other embodiments, the enzyme-polymer complex is a DNA polymerase-DNA template complex that is attached to an oligomeric nanopore, which can be a homo-oligomeric or a hetero-oligomeric nanopore, e.g., an aHL nanopore, which may be wild-type, a variant, or a modified variant nanopore.

### Forming Bilayers on Biochips

Methods for creating lipid bilayers comprising nanopores in a biochip are described at least at paragraphs [00131]-[00196] in PCT/US2014/061854 (published as WO2015/061511, Genia Technologies, Inc.). A lipid bilayer can be created on top of each one of multiple electrodes that make up an array of individually controlled electrodes and a single nanopore can be inserted into each bilayer atop each electrode in an array of individually controlled electrodes.

In some embodiments, sequencing nanopore complexes are formed on a semiconductor chip having multiple electrodes, to which a lipid solution is applied to create a lipid bilayer. The lipid solution may be a solution of an organic solvent, e.g., decane, hexane, tridecane etc., and lipid molecules, such as diphytanoylphosphatidylcholine (DPhPC), 1,2-diphytanoyl-sn-glycero-3-phosphocholine, Lysophosphatidylcholine (LPC), 1,2-Di-O-Phytanyl-sn-Glycero-3-phosphocholine (DoPhPC), palmitoyl-oleoyl-phosphatidyl-choline (POPC), dioleoyl-phosphatidyl-methylester (DOPME), dipalmitoylphosphatidylcholine (DPPC), phosphatidylcholine, phosphatidylethanolamine, phosphatidylserine, phosphatidic acid, phosphatidylinositol, phosphatidylglycerol, sphingomyelin, 1,2-di-O-phytanyl-sn-glycerol; 1,2-dipalmitoyl-sn-glycero-3-phosphoethanolamine-N-[methoxy(polyethylene glycol)-350]; 1,2-dipalmitoyl-sn-glycero-3-phosphoethanolamine-N-[methoxy(polyethylene glycol)-550]; 1,2-dipalmitoyl-sn-glycero-3-phosphoethanolamine-N-[methoxy(polyethylene glycol)-750]; 1,2-dipalmitoyl-sn-glycero-3-phosphoethanolamine-N-[methoxy(polyethylene glycol)-1000]; 1,2-dipalmitoyl-sn-glycero-3-phosphoethanolamine-N-[methoxy(polyethylene glycol)-2000]; 1,2-dioleoyl-sn-glycero-3-phosphoethanolamine-N-lactosyl; GM1 Ganglioside, or any combination thereof. Methods for applying the lipid solution and forming the lipid bilayer is described at least at paragraphs [00148] to [00152] of WO2015/061510.

### Inserting a Nanopore

In some instances, a nanopore is inserted in the membrane (e.g., by electroporation). Methods for inserting a nanopore in a membrane are provided at least at paragraphs [00197]-[00203] of WO2015/061510. The nanopore can be inserted by a stimulus signal such as electrical stimulus, pressure stimulus, liquid flow stimulus, gas bubble stimulus, sonication, sound, vibration, or any combination thereof.

In some embodiments, inserting the nanopore comprises applying a stimulus (e.g., electroporation pulse) through the electrode(s) to facilitate the insertion of said nanopore. In some cases, this is followed by a second electrical detection pulse to detect the insertion of said nanopore in said lipid bilayer. In other embodiments, the nanopore inserts itself into the membrane.

### Biochips

Nanopores of the nanopore sequencing complexes described herein may be inserted in a membrane, e.g. a lipid bilayer, and disposed adjacent or in proximity to a sensing electrode of a sensing circuit, such as an integrated circuit of a nanopore based sensor, e.g., a biochip. The nanopore may be inserted in a membrane and disposed of a well and/or sensing electrodes in the biochip. Multiple nanopore sensors may be provided as arrays. Biochips and methods for making biochips are described in PCT/US2014/061854 (published as WO2015/061511, Genia Technologies, Inc.), which is herein incorporated by reference in its entirety.

For embodiments that include an array of nanopores in a membrane, e.g., lipid bilayer, the density of sequencing nanopore complexes can be high. High density arrays are characterized as having a membrane surface that has a density of nanopore sequencing complexes greater or equal to about to about 500 nanopore sequencing complexes per 1 mm². In some embodiments, the surface has a density of discrete nanopore sequencing complexes of about 100, about 200, about 300, about 400, about 500, about 600, about 700, about 800, about 900, about 1000, about 2000, about 3000, about 4000, about 5000, about 6000, about 7000, about 8000, about 9000, about 10000, about 20000, about 40000, about 60000, about 80000, about 100000, or about 500000 nanopore sequencing complexes per 1 mm². In some embodiments, the surface has a density of discrete nanopore sequencing complexes of at least about 200, at least about 300, at least about 400, at least about 500, at least about 600, at least about 700, at least about 800, at least about 900, at least about 1000, at least about 2000, at least about 3000, at least about 4000, at least about 5000, at least about 6000, at least about 7000, at least about 8000, at least about 9000, at least about 10000, at least about 20000, at least about 40000, at least about 60000, at least about 80000, at least about 100000, or at least about 500000 nanopore sequencing complexes per 1 mm².

### Methods for Sequencing

The molecules being characterized using the nanopores of the nanopore sequencing complexes described herein can be of various types, including charged or polar molecules such as charged or polar polymeric molecules. Specific examples include ribonucleic acid (RNA) and deoxyribonucleic acid (DNA) molecules. The DNA can be a single-strand DNA (ssDNA) or a double-strand DNA (dsDNA) molecule. Other polymers that can be sequenced include proteins and peptides.

In one aspect, provided are methods for sequencing nucleic acids using the nanopore sequencing complexes described herein. The methods comprise (a) preparing an enzyme-polymer complex, and attaching the enzyme-polymer complex to a nanopore that is inserted into a membrane to provide a nanopore sequencing complex. The nanopore sequencing complexes prepared as described herein can be used for determining the sequence of nucleic acids according to other nanopore sequencing platforms known in the art that utilize enzymes in the sequencing of polynucleotides. For example, nanopore sequencing complexes can be prepared according to the method described herein for sequencing nucleic acids according to the helicase and exonuclease-based methods of Oxford Nanopore (Oxford, UK), Illumina (San Diego, Calif.), and the nanopore sequencing-by-expansion of Stratos Genomics (Seattle, Wash.).

In some embodiments, sequencing of nucleic acids comprises preparing nanopore sequencing complexes as described herein, and determining polynucleotide sequences using tagged nucleotides as is described in PCT/US2013/068967 (entitled “Nucleic Acid Sequencing Using Tags” filed on Nov. 7, 2013, which is herein incorporated by reference in its entirety). For example, a nanopore sequencing complex that is situated in a membrane (e.g., a lipid bilayer) adjacent to or in sensing proximity to one or more sensing electrodes, can detect the incorporation of a tagged nucleotide by a polymerase as the nucleotide base is incorporated into a strand that is complementary to that of the polynucleotide associated with the polymerase, and the tag of the nucleotide is detected by the nanopore. The polymerase-polynucleotide complex can be associated with the nanopore as described above.

Tags of the tagged nucleotides can include chemical groups or molecules that are capable of being detected by a nanopore. Examples of tags used to provide tagged nucleotides are described at least at paragraphs [0414] to [0452] of PCT/US2013/068967. Nucleotides may be incorporated from a mixture of different nucleotides, e.g., a mixture of tagged dNTPs where N is adenosine (A), cytidine (C), thymidine (T), guanosine (G) or uracil (U). Alternatively, nucleotides can be incorporated from alternating solutions of individual tagged dNTPs, i.e., tagged dATP followed by tagged dCTP, followed by tagged dGTP, etc. Determination of a polynucleotide sequence can occur as the nanopore detects the tags as they flow through or are adjacent to the nanopore, as the tags reside in the nanopore and/or as the tags are presented to the nanopore. The tag of each tagged nucleotide can be coupled to the nucleotide base at any position including, but not limited to a phosphate (e.g., gamma phosphate), sugar or nitrogenous base moiety of the nucleotide. In some cases, tags are detected while tags are associated with a polymerase during the incorporation of nucleotide tags. The tag may continue to be detected until the tag translocates through the nanopore after nucleotide incorporation and subsequent cleavage and/or release of the tag. In some cases, nucleotide incorporation events release tags from the tagged nucleotides, and the tags pass through a nanopore and are detected. The tag can be released by the polymerase, or cleaved/released in any suitable manner including without limitation cleavage by an enzyme located near the polymerase. In this way, the incorporated base may be identified (i.e., A, C, G, T or U) because a unique tag is released from each type of nucleotide (i.e., adenine, cytosine, guanine, thymine or uracil). In some situations, nucleotide incorporation events do not release tags. In such a case, a tag coupled to an incorporated nucleotide is detected with the aid of a nanopore. In some examples, the tag can move through or in proximity to the nanopore and be detected with the aid of the nanopore.

In some cases, tagged nucleotides that are not incorporated pass through the nanopore. The method can distinguish between tags associated with un-incorporated nucleotides and tags associated with incorporated nucleotides based on the length of time the tagged nucleotide is detected by the nanopore. In one embodiment, an un-incorporated nucleotide is detected by the nanopore for less than about 1 millisecond and an incorporated nucleotide is detected by the nanopore for at least about 1 millisecond.

Thus, in one aspect, the disclosure provides for a method for sequencing a polynucleotide from a biological sample, with the aid of a nanopore sequencing complex. The sample polynucleotide is combined with a polymerase, to provide the enzyme-polymer complex portion of the nanopore sequencing complex. In one embodiment, the sample polynucleotide is a sample ssDNA strand, which is combined with a DNA polymerase to provide a DNA polymerase-DNA complex. The DNA polymerase-sample ssDNA strand is subsequently attached to a nanopore that has been inserted into a membrane, e.g., a lipid bilayer, to provide the nanopore sequencing complex. The nanopore portion of the sequencing complex is positioned in the membrane adjacent to or in proximity of a sensing electrode, as described elsewhere herein. The resulting nanopore sequencing complex is capable of determining the sequence of nucleotide bases of the sample DNA as described elsewhere herein.

In one embodiment, the method provides for sequencing a sample polynucleotide with the aid of a nanopore sequencing complex adjacent to a sensing electrode, and comprises (a) preparing a nanopore sequencing complex by attaching an enzyme-polynucleotide complex, e.g., a DNA polymerase-DNA complex, to a nanopore that is inserted in a membrane, e.g., a lipid bilayer; (b) providing tagged nucleotides into a reaction chamber comprising the nanopore sequencing complex, wherein an individual tagged nucleotide of the tagged nucleotides contains a tag coupled to a nucleotide, which tag is detectable with the aid of the nanopore; (c) carrying out a polymerization reaction, with the aid of a polymerase, thereby incorporating an individual tagged nucleotide of the tagged nucleotides into a growing strand complementary to the single stranded nucleic acid molecule from the nucleic acid sample; and (d) detecting, with the aid of the nanopore, a tag associated with the individual tagged nucleotide during and/or upon incorporation of the individual tagged nucleotide, wherein the tag is detected with the aid of the nanopore when the nucleotide is associated with the polymerase. Other embodiments of the sequencing method that comprise the use of tagged nucleotides with the present nanopore sequencing complexes for sequencing polynucleotides are provided in WO2014/074727, which is incorporated herein by reference in its entirety.

Sequencing nucleic acids using AC waveforms and tagged nucleotides is described in US Patent Publication US2014/0134616 entitled “Nucleic Acid Sequencing Using Tags”, filed on Nov. 6, 2013, which is herein incorporated by reference in its entirety. In addition to the tagged nucleotides described in US2014/0134616, sequencing can be performed using nucleotide analogs that lack a sugar moiety or comprise an acyclic moiety, e.g., (S)-Glycerol nucleoside triphosphates (gNTPs) of the five common nucleobases: adenine, cytosine, guanine, uracil, and thymidine (Horhota et al. Organic Letters, 8:5345-5347 [2006]).

### EXAMPLES

### Example 1

This example shows that a nanopore complex comprising a nanopore, a polymerase and a polynucleotide template can be formed by attaching the polymerase-template complex to a nanopore that is inserted into a lipid bilayer.

A DNA template was bound to a mutant variant DNA polymerase comprising a SpyCatcher epitope in a ratio of 2 template:1 polymerase. Binding of the template to the polymerase was allowed to proceed for 30 minutes at 4° C.

A 1:6 aHL nanopore (1% solution) wherein one of the seven monomers in each complex contains one (1) SpyTag epitope (SEQ ID NO:2) and the other six monomers do not contain a spytag epitope (SEQ ID NO: 1) was electroporated into a lipid bilayer above the chip.

A mixture of template-bound polymerase and tagged nucleotides where the tag is a polymer of 30 thymine nucleotides (T30) consisting of 3 uM T-T30, 3 uM C-T30, 3 uM G-T30, and 3 uM A-T30, in static conditions (500 mM KGlu, 3 mM CaCl₂, 20 mM HEPES, pH8.0), was flowed over the nanopores inserted in the lipid bilayer at a rate of 0.834 ul/second.

The mixture was incubated for 30 minutes to allow for binding of the Spycatcher epitope on the polymerase-template complex to the SpyTag on the nanopore. Additional tagged T30-nucleotides were then flowed onto the pore-polymerase-template complex in the lipid bilayer.

An alternating current of 210 mV peak to peak was applied at 25 Hz, and capture of nucleotide tags was assessed as nucleotide bases were incorporated into the copied DNA strand by the nanopore-bound polymerase.

The current trace shown in FIG. 3 demonstrates that nucleotide tags were detected by the nanopore complex formed according to the method provided herein.

SEQUENCE LISTING FREE TEXT

| SEQ ID NO: 1 (aHL polypeptide; pA020 HemoM H144A) | |
|---|---|
| MADSDINIKTGTIDIGSNTIVKTGDLVTYDKENGMHKKVFYSFIDDKNH | |
| NKKLLVIRTKGTIAGQYRVYSEEGANKSGLAWPSAFKVQLQLPDNEVAQ | |
| ISDYYPRNSIDTKEYMSTLTYGFNGNVTGDDTGKIGGLIGANVSIGATLKY | |
| VQPDFKTILESPTDKKVGWKVIFNNMVNQNWGPYDRDSWNPVYGNQ | |
| LFMKTRNGSMKAADNFLDPNKASSLLSSGFSPDFATVITMDRKASKQQT | |
| NIDVIYERVRDDYQLHWTSTNWKGTNTKDKWTDRSSERYKIDWEKEEM | |
| TNGLSAWSHPQFEK | |
| SEQ ID NO: 2 (aHL polypeptide; pA018: Hemolysin Spytag-Histag) | |
| MADSDINIKTGTIDIGSNTIVKTGDLVTYDKENGMHKKVFYSFIDDKNH | |
| NKKLLVIRTKGTIAGQYRVYSEEGANKSGLAWPSAFKVQLQLPDNEVAQ | |
| ISDYYPRNSIDTKEYMSTLTYGFNGNVTGDDTGKIGGLIGANVSIGHTLKY | |
| VQPDFKTILESPTDKKVGWKVIFNNMVNQNWGPYDRDSWNPVYGNQLF | |
| MKTRNGSMKAADNFLDPNKASSLLSSGFSPDFATVITMDRKASKQQTN | |
| IDVIYERVRDDYQLHWTSTNWKGTNTKDKWTDRSSERYKIDWEKEEM | |
| TNGGSSGGSSGGAHIVMVDAYKPTKKGHHHHHH | |
| SEQ ID NO: 3 (DNA polymerase polypeptide; Pol6-2 NL) | |
| MHHHHHHHHSGDYDIPTTENLYFQGAMVDTLSGLSSEQGQSGDMTIEED | |
| SATHI KFSKRDEDGKELAGATMELRDSSGKTISTWISDGQVKDFYL | |
| YPGKYTFVET | |
| AAPDGYEVATAITFTVNEQGQVTVNGKATKGDAHIGGSDKHTQYVKEHS | |
| FNYDEYKKANFDKIECLIFDTESCTNYENDNTGARVYGWGLGVTRNH | |
| NMIYGQN | |
| LNQFWEVCQNIFNDWYHDNKHTIKITKTKKGFPKRKYIKFPIAVHNLGWDV | |
| EFLKYSLVENGFNYDKGLLKTVFSKGAPYQTVTDVEEPKTFHIVQNNNI | |
| VYGCNVYMDKFFEVENKDGSTTEIGLCLDFFDSYKIITCAESQFHNYV | |
| HDVDPMFYKMGEEYDYDTWRSPTHKQTILELRYQYNDIYMLREVIEQ | |
| FYIDGLCGGELPLTGMRTASSIAFNVLKKMTFGEEKTEEGYINYFELDKK | |
| TKFEFLRKRIEMESYTGGYHANHKAVGKTINKIGCSLDINSAYPSQMAYK | |
| VFPYGKPVRKTWGRKPKTEKNEVYLIEVGFDFVEPKHEEYALDIFKIG | |
| AVNSKALSPITGAVSGQEYFCTNIKDGKAIPVYKELKDTKLTINYNWLT | |
| SVEYEFWIKHFNFGVFKKDEYDCFEVDNLEFTGLKIGSILYYKAEKGKF | |
| KPYVDHFTKMKVELKKLGNKPLTNQAKLILNGAYGKFGTKQNKEEKDL | |
| IMDKNGLLTFTGSVTEYEGKEFYRPYASFVTAYGRLQLWNAIIYAVGV | |
| ENFLYCDTDSIYCNREVNSLIEDMNAIGETIDKTQLGKWDVEHVFDKFK | |
| VLGQKKYMYHDCKEDKTDLKCCGLPSDARKIIIGQGFDEFYLGKNVEGK | |
| KQRKKVIGGCLLLDTLFTIKKIMF | |
| SEQ ID NO: 4 (DNA polymerase polypeptide; Pol6 wild- | |
| type[Clostridium phage phiCPV4]; GenBank: AFH27113.1) | |
| 1 mdkhtqyvke hsfnydeykk anfdkiecli fdtesctnye ndntgarvyg | |
| wglgvtrnhn 061 miygqnlngf wevcqnifnd | |
| wyhdnkhtik itktkkgfpk rkyikfpiav hnlgwdvefl 121 kyalvengfn | |
| ydkgllktvf skgapyqtvt dveepktfhi vqnnnivygo nvymdkffev 181 | |
| enhdgsttei glcldffdey kiitcaesqf hnyvhdvdpm fykmgeeydy dtwrapthkq | |
| 241 ttlelryqyn diymlrevie qfyidgicgg elpltgmrta ssiafnvlkk | |
| mcfgeektee 301 gyinyfeldk ktkfeflrkr ismesytggy thanhkavgk | |
| tinkigcald inasypagma 361 ykvfpygkg rktwgrkpkt sknevyliev | |
| gfdfvepkhe eyaldifkig aynskalspi 421 tgavsgqeyf ctnikdgkai | |
| pvykelkdtk lttnynvvlt sveyafwikh fnfgvfkkde 481 ydcfevdnle | |
| ftglkigsil yykaekgkfk pyvdhftkmk venkklgnkp ltnqakliln 541 | |
| gaygkfgtkq nkeekdlimd knglltftgs vteyegkefy rpyasfvtay grlqlwnaii | |
| 601 yavgvenfly cdtdaiycnr evneliedmn aigetidkti lgkwdvehvf | |
| dkfkvlgqkk 661 ymyhdckedk tdlkccglps darkiiigqg fdefylgknv | |
| egkkqrkkvi ggcllldtlf 721 tikkimf | |
| SEQ ID NO: 5 (Mature WT aHL; AAA26598) | |
| ADSDINIKTG TTDIGSNTTV KTGDLVTYDK ENGMHKKVFY SFIDDKNHNK | 50 |
| KLLVIRTKGT IAGQYRVYSE EGANKSGLAW PSAFKVQLQL PDNEVAQISD | 100 |
| YYPRNSIDTK EYMSTLTYGF NGNVTGDDTG KIGGLIGANV SIGHTLKYVQ | 150 |
| PDFKTILESP TDKKVGWKVI FNNMVNQNWG PYDRDSWNPV YGNQLFMKTR | 200 |
| NGSMKAADNF LDPNKASSLL SSGFSPDFAT VITMDRKASK QQTNIDVIYE | 250 |
| RVRDDYQLHW TSTNWKGTNT KDKWTDRSSE RYKIDWEKEE MTN | 293 |

### CITATION LIST

### Non-Patent Literature

[1] Venkatesan and Bashir, “Nanopore Sensors for Nucleic acid Analysis”, Nature Biotechnol, DOI:10.1038/NNANA.2011.129 [2011].
[2] Watson, J. D. et al. In: Molecular Biology of the Gene, 4th Ed., W. A. Benjamin, Inc., Menlo Park, Calif. (1987).
[3] Astier et al., “Toward single molecule DNA sequencing: direct identification of ribonucleoside and deoxyribonucleoside 5-monophosphates by using an engineered protein nanopore equipped with a molecular adapter”, J Am Chem Soc 128:1705-1710 [2006].
[4] Nivala et al., “Unfoldase-mediated protein translocation through an a-hemolsyin nanopore”, Nature Biotechnol 31:247-250 [2013].
[5] Zakeri et al. “Peptide tag forming a rapid covalent bond to a protein, through engineering a bacterial adhesin”, PNAS 109:E690-E697 [2012].
[6] Thapa et al., “Native Chemical Ligation”: A Boon to Peptide Chemistry”, Molecules 19:14461-14483 [2014].
[7] Wu and Guo, “Sortase-Mediated Transpeptidation for Sit-Specific Modification of Peptides, Glycopeptides, and Proteins”, J Carbohydr Chem 31:48-66 [2012].
[8] Heck et al., “Enzyme-catalyzed protein crosslinking”, Appl Microbiol Biotechnol 97:461-475 [2013].
[9] Dennler et al., “Transglutaminase-Based Chemo-Enzymatic Conjugation Approach Yields Homogeneous Antibody-Drug Conjugates”, Bioconjug Chem 25:569-578 [2014].
[10] Rashidian et al. “Chemoenzymatic Labeling of Proteins: Techniques and Approaches”, Bioconjug Chem 24:1277-1294 120131.
[11] Horhota et al. Organic Letters, 8:5345-5347 [2006].

### PATENT LITERATURE

[12] PCT/US2013/068967 (published as WO2014/074727 on 15 May 2014; Genia Technologies; entitled NUCLEIC ACID SEQUENCING USING TAGS).
[13] PCT/US2005/009702 (published as WO2006/028508 on 16 Mar. 2006; President and Fellows of Harvard College; entitled METHODS AND APPARATUS FOR CHARACTERIZING POLYNUCLEOTIDES.
[14] PCT/US2011/065640 (published as WO2012/083249; Columbia University; entitled DNA SEQUENCING BY SYNTHESIS USING MODIFIED NUCLEOTIDES AND NANOPORE DETECTION).
[15] PCT/US2014/061854 (published as WO2015/061511, Genia Technologies, Inc.; entitled PROCESS FOR BIOSENSOR WELL FORMATION).
[16] PCT/US2014/061853 (published as WO2015/061510, Genia Technologies, Inc.; entitled METHODS FOR FORMING LIPID BILAYERS ON BIOCHIPS). U.S. patent application Ser. No. 14/073,445 (published as US2014/0134616, Genia Technologies, Inc.; entitled NUCLEIC ACID SEQUENCING USING TAGS

1-15. (canceled)

16. A method for preparing a nanopore sequencing complex, the method comprising:
(a) inserting a nanopore into a membrane;
(b) contacting the nanopore with a sequencing enzyme;
(c) attaching the enzyme to the nanopore to form an enzyme-nanopore complex; and
(d) binding a polymer to the enzyme-nanopore complex to provide a nanopore sequencing complex.

17. The method of claim 16, wherein the sequencing enzyme of is a polymerase, an exonuclease, a helicase, or an unfoldase.

18. The method of claim 17, wherein the polymerase is a DNA polymerase, a reverse transcriptase, or an RNA polymerase.

19. The method of claim 17, wherein the polymerase is a wild-type or variant thereof.

20. The method of claim 19, wherein the variant polymerase has increased enzyme activity, fidelity, processivity, elongation rate, stability, or solubility.

21. The method of claim 16, wherein the nanopore is selected from a monomeric nanopore, a homo-oligomeric nanopore, and a hetero-oligomeric nanopore.

22. The method of claim 16, wherein the nanopore comprises an a-hemolysin nanopore (αHL), and outer membrane protein G nanopore (OmpG), or a variant or modified variant thereof.

23. A method for sequencing a nucleic acid sample, comprising:
(a) preparing a nanopore sequencing complex in a lipid bilayer according to claim 1;
(b) providing tagged nucleotides or nucleotide analogs to the nanopore sequencing complex, wherein the tag of the tagged nucleotide is detectable with the aid of the nanopore;
(c) carrying out a polymerization reaction with the aid of the polymerase coupled to the nanopore in the nanopore-sequencing complex, thereby incorporating an individual tagged nucleotide of the tagged nucleotides into a growing strand complementary to a sample ssDNA template; and
(d) detecting, with the aid of the nanopore, a tag associated with the individual tagged nucleotide during incorporation of the individual tagged nucleotide, wherein the tag is detected with the aid of the nanopore while the nucleotide is associated with the polymerase, thereby providing a sequence of the nucleic acid sample.

24. A biochip for sequencing a nucleic acid sample, the biochip comprising: an array of a plurality of nanopore sequencing complexes disposed in a membrane and prepared according to claim 1, a nanopore of the plurality being disposed adjacent or in proximity to an electrode, wherein the nanopore is individually addressable and has a single polymerase-polynucleotide complex attached to the nanopore; and wherein an individual nanopore detects the tag associated with the tagged nucleotide during incorporation of the nucleotide into a growing nucleic acid chain by the polymerase.

25. A method for preparing a nanopore sequencing complex, the method comprising:
(a) inserting a nanopore into a membrane;
(b) combining a sequencing enzyme with a polymer to provide an enzyme-polymer complex; and
(c) attaching the enzyme-polymer complex to the pore, thereby providing a nanopore sequencing complex.

26. A method according to claim 25, wherein the enzyme-polymer complex is polymerase-polynucleotide complex.

27. The method of claim 25, wherein the enzyme-polymer complex attached to the nanopore is an interchangeable enzyme-polymer complex that can be substituted with any one of a plurality of different enzyme-polymer complexes.

28. The method of claim 27, wherein the interchangeable enzyme-polymer complex is an interchangeable polymerase-polynucleotide complex that is substituted with any one of a plurality of different polymerase-polynucleotide complexes.

29. The method of claim 28, wherein the polymerase-polynucleotide complex is selected from a group consisting of a DNA polymerase-DNA complex, a reverse-transcriptase-RNA complex, and an RNA polymerase-RNA complex.

30. The method of claim 25, wherein the sequencing enzyme of is a polymerase, an exonuclease, a helicase, or an unfoldase.

31. The method of claim 30 wherein the polymerase is a DNA polymerase, a reverse transcriptase, or an RNA polymerase.

32. The method of claim 30, wherein the polymerase is a wild-type or variant thereof.

33. The method of claim 32, wherein the variant polymerase has increased enzyme activity, fidelity, processivity, elongation rate, stability, or solubility.

34. The method of claim 26, wherein the polynucleotide of the polymerase-polynucleotide complex comprises a single stranded DNA (ssDNA) template sample, a double stranded DNA (dsDNA) sample, or an RNA sample.

35. The method of claim 25, wherein the nanopore is selected from a monomeric nanopore, a homo-oligomeric nanopore, and a hetero-oligomeric nanopore.

36. The method of claim 35, wherein the nanopore comprises an a-hemolysin nanopore (αHL), Outer membrane protein G nanopore (OmpG), or a variant or modified variant thereof.

37. A method for sequencing a nucleic acid sample, the method comprising:
(a) preparing a nanopore sequencing complex in a lipid bilayer according to claim 25;
(b) providing tagged nucleotides or nucleotide analogs to the nanopore sequencing complex, wherein the tag of the tagged nucleotide is detectable with the aid of the nanopore;
(c) carrying out a polymerization reaction with the aid of the polymerase coupled to the nanopore in the nanopore-sequencing complex, thereby incorporating an individual tagged nucleotide of the tagged nucleotides into a growing strand complementary to a sample ssDNA template; and
(d) detecting, with the aid of the nanopore, a tag associated with the individual tagged nucleotide during incorporation of the individual tagged nucleotide, wherein the tag is detected with the aid of the nanopore while the nucleotide is associated with the polymerase, thereby providing a sequence of the nucleic acid sample.

38. A biochip for sequencing a nucleic acid sample, the biochip comprising: an array of a plurality of nanopore sequencing complexes disposed in a membrane and prepared according to claim 25, a nanopore of the plurality being disposed adjacent or in proximity to an electrode, wherein the nanopore is individually addressable and has a single polymerase-polynucleotide complex attached to the nanopore; and wherein an individual nanopore detects the tag associated with the tagged nucleotide during incorporation of the nucleotide into a growing nucleic acid chain by the polymerase.
